(19) United States
(12) Patent Application Publication
(10) Pub. No.: US 2021/0366595 A1
(43) Pub. Date: Nov. 25, 2021

(54) MANAGEMENT SYSTEM

(71) Applicant: FUJIFILM Corporation, Tokyo (JP)

(72) Inventors: Kazuhiro HIROTA, Ashigarakami-gun (JP); Yoshihiro SETO, Ashigarakami-gun (JP); Kaku IRISAWA, Ashigarakami-gun (JP)

(73) Assignee: FUJIFILM Corporation, Tokyo (JP)

(21) Appl. No.: 17/393,662

(22) Filed: Aug. 4, 2021

Related U.S. Application Data
(63) Continuation of application No. PCT/JP2020/003389, filed on Jan. 30, 2020.

(30) Foreign Application Priority Data
Feb. 5, 2019 (JP) 2019-019058

(51) Int. Cl. G16H 30/40; G16H 30/20; G16H 40/20

(52) U.S. Cl. CPC G16H 30/40; G16H 40/20; G16H 30/20

(57) ABSTRACT

A management system includes a label imaging unit that images labels of a plurality of sample containers, a label recognition unit that recognizes a subject by using label images, a test order acquisition unit that acquires a test order of the subject, an automatic association unit that automatically associates a test result related to the sample of each sample container with the test order acquired by the test order acquisition unit by using a recognition result of the label recognition unit, and a display unit that displays the test result and the test order in association with each other.

### CROSS-REFERENCE TO RELATED APPLICATIONS

This application is a Continuation of PCT International Application No. PCT/JP2020/003389 filed on 30 Jan. 2020, which claims priority under 35 U.S.C. § 119(a) to Japanese Patent Application No. 2019-019058 filed on 5 Feb. 2019. The above application is hereby expressly incorporated by reference, in its entirety, into the present application.

### BACKGROUND OF THE INVENTION

### 1. Field of the Invention

The present invention relates to a management system that associates a subject, a test result, a test order that is a request for a test, and the like.

### 2. Description of the Related Art

In the related art, various tests such as a fecal occult blood test or a blood test have been performed as needed for a health diagnosis and the like. Some of these tests are performed at a hospital, but the hospital usually requests a testing institution to perform the test. That is, the testing institution that actually performs the test may be a different institution from the hospital that uses test results.

For example, in the case of a fecal occult blood test, a subject collects a sample such as feces and submits it to a hospital or a testing institution. On the other hand, a doctor or the like in a hospital specifies a subject to the testing institution and requests the testing institution to perform a fecal occult blood test. Then, the testing institution performs a test on a sample of the subject or a specific subject provided by the hospital based on a test order received from the hospital, and notifies the hospital and/or the subject of the result.

Then, the testing institution is performing the work of associating the test result related to the sample in a sample container containing the sample with the test order. Specifically, in order to associate the test result with the subject, the testing institution associates the subject with the test result by giving an identification (ID) to each sample container using a barcode or the like and managing the test result. In addition, the sample container is usually submitted by the subject with a label having his/her name, gender, age, and the like described thereon, but since association with the test order is not performed, the test result and the test order are manually associated with each other at the testing institution. For example, by visually checking the label of the sample container and the name or the like registered in the test order, the test result output in association with the ID of the sample container is registered as the test result for the corresponding test order.

In recent years, in a case where an ID of a subject and a test result are output in association with each other from a test device and the subject is registered in a test order with the same ID, a management device that automatically associates the test result with the test order is known (JP2018-084862A). In addition, in systems used in private hospitals and the like, there is also known a system that specifies a subject to be tested next and automatically registers a test result output by a test device as a test result of the specified subject (JP2011-206557A).

### SUMMARY OF THE INVENTION

The work of associating a subject, a sample submitted by the subject, a container containing the sample, a test result, a test order, and the like is complicated. In particular, in a testing institution, there is a set of test orders for a plurality of subjects, and a plurality of sample containers should be handled at substantially the same time. Thus, efforts are required even just for giving an ID to the sample container and associating the test result with the subject information such as the name of the subject. In addition, a plurality of sets of subject information and test results that have been obtained should be compared with the test order, but in a case where the same ID as the test order can be used, or unless it is a special environment such as a small-scale test in which the test and the information of the subject can be associated one-to-one before the test is performed, the actual situation is that the test results and the subject information are associated with the test order while visually checking. Therefore, it is desired to improve the efficiency of the association work in the testing institution by efficiently performing the test on the samples for a plurality of persons and automatically performing the association work.

An object of the present invention is to provide a management system capable of automatically associating a plurality of test results with a plurality of test orders in a large-scale testing institution that should handle tests for a plurality of persons substantially at the same time.

According to an aspect of the present invention, there is provided a management system comprising a label imaging unit, a label recognition unit, a test order acquisition unit, an automatic association unit, and a display unit. The label imaging unit is configured to image labels attached to a plurality of sample containers each containing a sample, the label having subject information of a subject from whom the sample is collected described thereon, the label recognition unit is configured to recognize the subject from whom the sample contained in each of the sample containers is collected by using label images obtained by imaging the labels, the test order acquisition unit is configured to acquire a test order including the subject information of the subject from whom the sample is collected, the automatic association unit is configured to automatically associate a test result related to the sample of each sample container with the test order acquired by the test order acquisition unit by using a recognition result of the label recognition unit, and the display unit is configured to display the test result and the test order in association with each other according to association by the automatic association unit.

It is preferable that the label recognition unit recognizes a color of the label by using the label image, and the automatic association unit associates the same test order for two sample containers having the label images of different colors.

It is preferable that the label imaging unit images the labels of the plurality of sample containers together, and extracts a portion of the label of each sample container as the label image from an obtained image.

It is preferable that description of the label includes a subject name, gender, and/or age of the subject, and the label recognition unit recognizes the subject name, gender, and/or age described on the label.

It is preferable that the management system further comprises a conversion unit configured to convert a character or a symbol recognized by the label recognition unit into another character or symbol, the automatic association unit associates each test result with the test order by using a character or a symbol obtained by converting the recognition result of the label recognition unit by the conversion unit.

It is preferable that the conversion unit converts the character recognized by the label recognition unit into a character type used in the test order for a subject name.

It is preferable that the label recognition unit recognizes the subject by using the label image of the label handwritten by the subject.

It is preferable that the display unit highlights a display portion in which the test order and the test result related to the label image with poor recognition whose accuracy of a recognition process is equal to or less than a threshold value are associated with each other.

It is preferable that the display unit displays the label image for at least the subject recognized by using the label image with poor recognition.

It is preferable that a correction for the subject information of the subject recognized by using the label image with poor recognition is accepted.

It is preferable that in a case where the subject information of the subject is corrected, the automatic association unit automatically associates the test order with the test result by using the corrected subject information.

It is preferable that at least in a case where there is the label image with poor recognition, the automatic association unit presents a candidate for the test order that is able to be associated with the test result in addition to the test order that is automatically associated with the test result.

According to the management system of the present invention, it is possible to automatically associate a plurality of test results with a plurality of test orders in a large-scale testing institution that should handle tests for a plurality of persons substantially at the same time.

### BRIEF DESCRIPTION OF THE DRAWINGS

FIG. 1 is an explanatory diagram showing an outline of a test such as a fecal occult blood test.

FIG. 2 is a sample container for a fecal occult blood test.

FIG. 3 is an external perspective view of a fecal occult blood test device (a test device).

FIG. 4 is a sample container and a rack for holding the sample container.

FIG. 5 is a block diagram showing a configuration of a test device, a management device, and a management system.

FIG. 6 is a label image.

FIG. 7 is test data in which a label image and a value of a measured value which is a test result are associated with each other.

FIG. 8 is an order list in which each row is a test order.

FIG. 9 is a flowchart relating to a test.

FIG. 10 is a flowchart relating to association.

FIG. 11 is an example of test data output by a test device.

FIG. 12 is an example of associated data displayed on a display unit.

FIG. 13 is an example of associated data having a different display form.

FIG. 14 is an explanatory diagram showing an example of extracting individual label images from images captured of a plurality of sample containers.

FIG. 15 is a block diagram of a management system having a conversion unit according to a second embodiment.

FIG. 16 is an example of test data in a case where there is poor recognition.

FIG. 17 is a display example of associated data in a case where there is poor recognition.

### DESCRIPTION OF THE PREFERRED EMBODIMENTS

[First Embodiment]

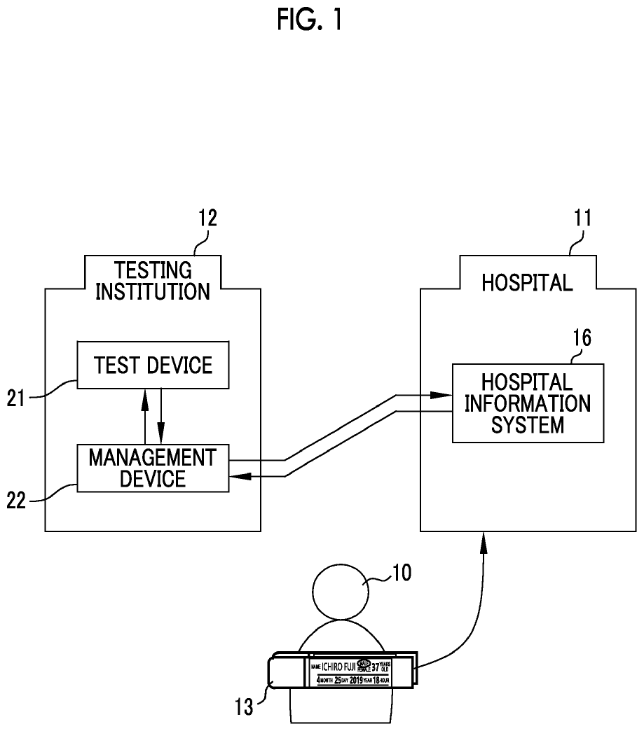

As shown in FIG. 1, in various medical-related tests such as a fecal occult blood test or a blood test (hereinafter, simply referred to as a “test”), a hospital 11 visited by a subject 10 for a health diagnosis, other tests, or diagnosis, and a testing institution 12 which is a separate organization from the hospital 11 are usually involved. In a case where a test is required, the subject 10 collects a sample to be used for the test based on the instruction of the doctor in charge at the hospital 11, contains the collected sample in a dedicated container (hereinafter, referred to as a sample container) 13, and submits it to the hospital 11. The sample is the body tissue or secretions of the subject 10. For example, in the fecal occult blood test, the sample is the feces of the subject 10. In the blood test, the sample is the blood of the subject.

On the other hand, the hospital 11 usually requests a test to the testing institution 12, which is a specialized institution for a test. For example, the hospital 11 issues a test request (hereinafter, referred to as a test order) to the testing institution 12 by using a hospital information system (HIS) 16 or the like. The test order includes at least information of a subject 10 who performs a test, that is, a subject 10 who collects a sample (hereinafter, referred to as subject information), and information indicating the target and contents of the test to be performed. Further, the hospital 11 sends the sample container 13 containing the sample to the testing institution 12.

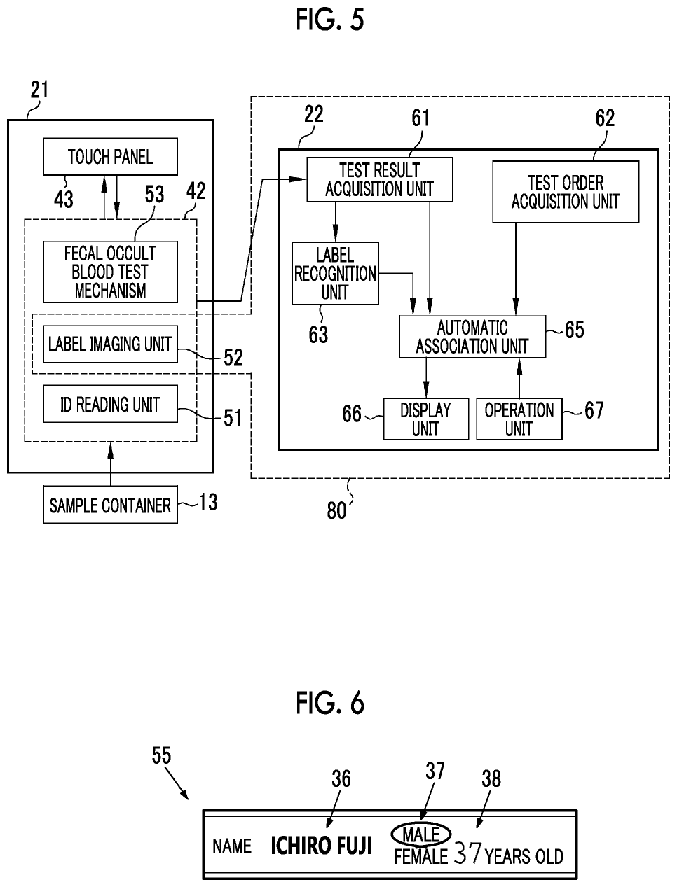

The testing institution 12 comprises a test device 21, and a management device 22. A management system 80 (see FIG. 5) of the present invention is a system composed of a part or all of the test device 21 and/or the management device 22, and associates information related to the subject 10 (hereinafter, referred to as subject information) with a test result and a test order. The subject information is an identification number such as the name or ID of the subject 10, gender, age, and the like, and includes at least one piece of information for distinguishing a specific subject 10 from other subjects 10.

The testing institution 12 may own one or a plurality of types of test devices 21 depending on the type of test to be performed. In a case where the testing institution 12 supports a fecal occult blood test and a blood test, the testing institution 12 comprises two types of test devices 21, a fecal occult blood test device and a blood test device. Further, the test device 21 may have a plurality of test devices 21 used for the specific test in order to perform the specific test substantially at the same time. For example, the testing institution 12 may have a plurality of fecal occult blood test devices, which are test devices 21.

The management device 22 manages the subject 10, the test result, the test order, and the like after associating the test results output by one or a plurality of test devices 21 with the subject 10 to be tested, the test result, and the test order, as described above. In addition, as necessary, the management device 22 may communicate with the hospital information system 16 and the like of the hospital 11 to transmit and receive necessary data. For example, the management device 22 reports the test result to the hospital 11 by transmitting or registering the test result to the hospital information system 16.

Hereinafter, for the sake of description, it is assumed that the test to be performed is specifically a fecal occult blood test. Therefore, the sample is the feces of the subject 10, and the sample container 13 is a container (a stool collection container) made into a kit so that the subject 10 can collect stool by himself/herself and store it stably for a certain period of time. The test device 21 is a fecal occult blood test device.

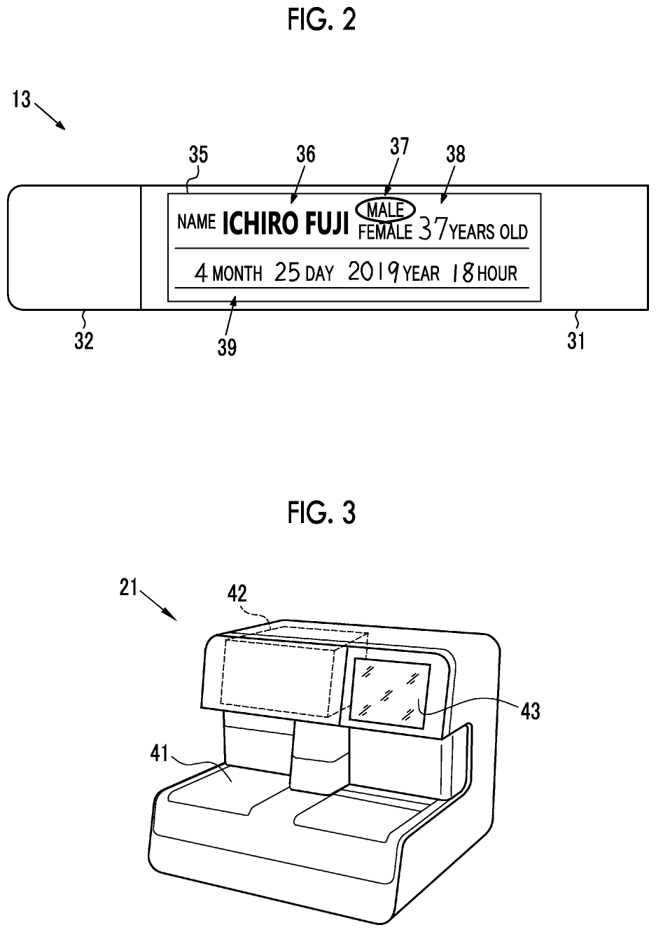

As shown in FIG. 2, the sample container 13 comprises a container body 31 for containing feces as a sample, and a cap 32 attachably and detachably provided on the container body 31. The container body 31 is, for example, transparent or translucent, and contains a storage solution of feces (or a diluent) as a sample therein. An insertion part (not shown) to be inserted into the container body 31 is attached to the cap 32. Therefore, the subject 10 removes the cap 32 from the container body 31, collects feces by tracing the surface of the feces as a sample with the distal end of the insertion part, inserts the insertion part into the container body 31, and closes the cap 32 to thereby collect the feces as a sample.

The sample container 13 is submitted to the hospital 11 with a label 35 attached to one surface of the container body 31. The subject information of the subject 10 from whom the sample is collected is described in the label 35. In the present embodiment, the label 35 has a name entry field 36, a gender selection field 37, an age entry field 38, and a stool collection date and time entry field 39. At least, the name of the subject 10 (“Ichiro Fuji” in FIG. 2) entered in the name entry field 36, the gender (“male” in FIG. 2) selected in the gender selection field 37, and the age (“37” years old in FIG. 2) entered in the age entry field 38 function as the subject information.

Further, the label 35 of the sample container 13 used in the fecal occult blood test has two types of labels 35 of different colors. Since the fecal occult blood test usually tests feces for two days, such labels 35 of different colors are used to distinguish the sample container 13 for these two days. Specifically, there are two types of labels 35, a first color label on which the characters indicating the name entry field 36 (“name”), the characters indicating the gender selection field 37 (“male” and “female”), the characters indicating the age entry field 38 (“years old”), the characters indicating the stool collection date and time entry field 39 (“month”, “day”, “year”, and “hour”), and/or other ruled lines are printed in a first color, and a second color label on which these are printed in a second color different from the first color. For example, the first color is a reddish color (red, orange, or the like), and the second color is a bluish color (blue, green, or the like).

As shown in FIG. 3, the test device 21 is a fecal occult blood test device, and comprises a sample container placing portion 41, a test unit 42 which is a substantive mechanism for performing a test, and a touch panel 43 which functions as an operation unit and a display unit of the test device 21.

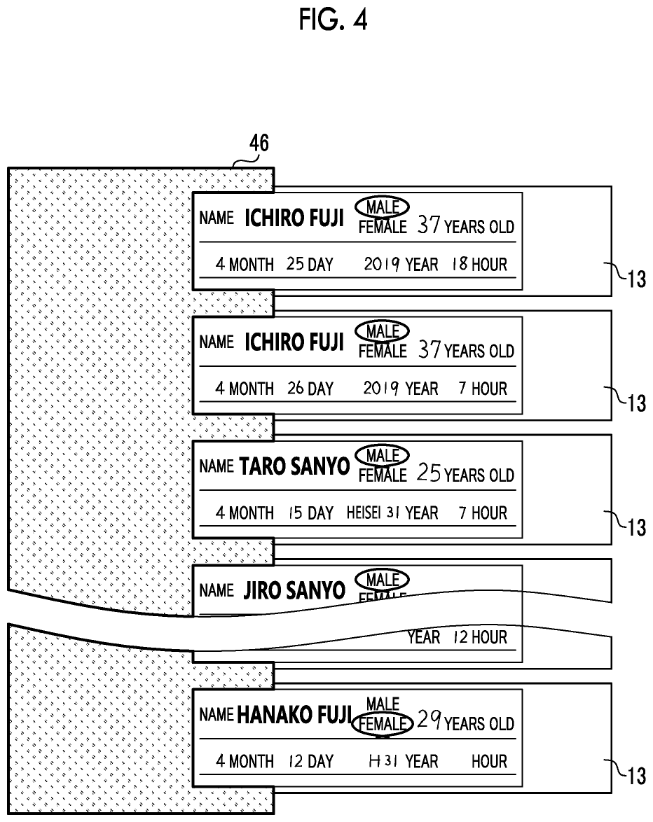

The sample container placing portion 41 is a portion on which one or a plurality of sample containers 13 are placed in a case of performing a test. In the present embodiment, as shown in FIG. 4, a plurality of sample containers 13 are set on the rack 46, which holds the respective labels 35 in an exposed state (a state where at least the portion related to the subject information is not hidden in the rack 46), with the surfaces to which the labels 35 are attached aligned. Then, the plurality of sample containers 13 are placed on the sample container placing portion 41 together with the rack 46. The sample container 13 set in the rack 46 is basically a sample container 13 submitted by different subjects 10. However, since the test in the present embodiment is a fecal occult blood test, two sample containers 13 submitted by the same subject 10 with the labels 35 of different colors may be set in one rack 46, which is permissible. In FIG. 4, the sample container 13 set on the leftmost side of the rack 46 and the sample container 13 set second from the left are the sample containers 13 for two days submitted by “Ichiro Fuji”, one of the subjects 10, respectively. The other sample containers 13 are those submitted by the subjects 10 other than at least “Ichiro Fuji”. An ID is given to the rack 46 by using a barcode, an IC chip, or the like (not shown), and the test device 21 individually recognizes the rack 46. This is to individually recognize the sample container 13 and the test result and to manage them without being confused with each other.

As shown in FIG. 5, the test unit 42 comprises an ID reading unit 51, a label imaging unit 52, and a fecal occult blood test mechanism 53. The ID reading unit 51 is, for example, a scanner that reads information (ID or the like) for identifying the rack 46 or the sample container 13. In the present embodiment, the ID reading unit 51 is a barcode reader that scans the barcode attached to the rack 46 to acquire the ID of the rack 46.

The label imaging unit 52 images the label 35 attached to the sample container 13, the label 35 having subject information described thereon. Specifically, the label imaging unit 52 can individually image the labels 35 of the plurality of sample containers 13 set in the rack 46. In addition, the label imaging unit 52 can image some or all of the labels 35 of the plurality of sample containers 13 set in the rack 46 together. The label imaging unit 52 images the label 35 of the sample container 13 at least by the time the management device 22 uses the test result output by the test device 21 (until the test result is used for associating with the test order). The label imaging unit 52 outputs a captured image 55 of the label 35 (hereinafter, referred to as a label image; see FIG. 6) to the management device 22. This is for the management device 22 to use the label image 55 for the association of the subject 10, the test result, and the test order. The label image 55 includes at least one subject information described on the label 35. In the present embodiment, as shown in FIG. 6, the label image 55 is an image showing the name entry field 36, the gender selection field 37, and the age entry field 38 of the label 35.

In the present embodiment, the plurality of sample containers 13 are set in the test device 21 at the same time using the rack 46, but the samples contained in each sample container 13 are sequentially tested by the fecal occult blood test mechanism 53. Then, the label imaging unit 52 images the label 35 of the sample container 13 at the timing of providing the sample contained in each sample container 13 to the fecal occult blood test mechanism 53, and obtains the label image 55. The test unit 42 outputs the test result of the sample contained in a specific sample container 13 and the label image 55 of the specific sample container 13 to the management device 22 in a one-to-one correspondence.

The fecal occult blood test mechanism 53 is a mechanism for performing a fecal occult blood test, and includes a stock of reagents used for the fecal occult blood test. In a case where the test is completed, the fecal occult blood test mechanism 53 sequentially outputs the results to the touch panel 43 and the management device 22. The touch panel 43 indicates the progress of the test by appropriately displaying the test results in the order in which the test is completed. Further, in a case where the test results are acquired from the fecal occult blood test mechanism 53, the management device 22 associates the subject 10, the test result, the test order, and the like sequentially or unit by unit (for example, for each rack 46).

The management device 22 is a so-called computer provided with a test result acquisition unit 61, a test order acquisition unit 62, a label recognition unit 63, an automatic association unit 65, a display unit 66, an operation unit 67, and the like (see FIG. 5).

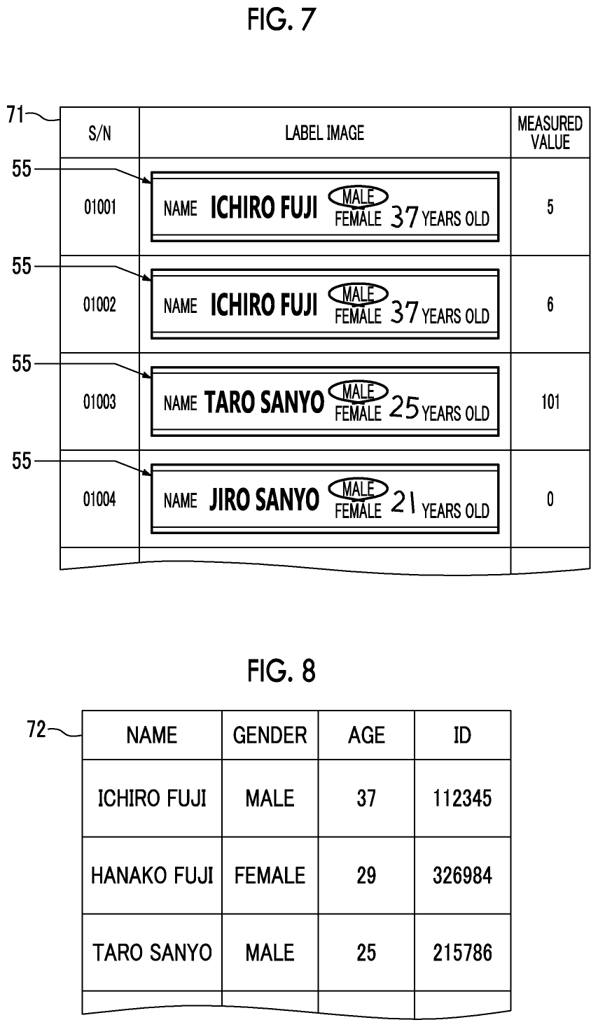

The test result acquisition unit 61 acquires the test result and the label image 55 from the test unit 42 of the test device 21. As described above, in the present embodiment, since the test unit 42 outputs the test result and the label image 55 in a one-to-one correspondence, the test result acquisition unit 61 acquires the test result and label image 55 corresponding to each other at the same time. That is, as shown in FIG. 7, the test result acquisition unit 61 acquires at least test data 71 in which the label image 55 and a value of a “measured value” which is the test result are associated with each other. In FIG. 7, the test data 71 includes serial number (S/N) data in addition to the label image 55 and the test result. The “serial number” is a numerical value representing the order in which the tests were performed (or the order in which the test results were acquired), and, for example, in a case where the test result acquisition unit 61 acquires the label image 55 and the test result (the measured value) corresponding to each other, the serial number which is an acquisition order is attached to a set of the label image 55 and the test result.

The test order acquisition unit 62 acquires a test order from, for example, the hospital information system 16. In the present embodiment, it is assumed that the test order acquired by the test order acquisition unit 62 includes the ID of the subject 10 (the ID used for management at the hospital 11), the name, gender, and age of the subject 10, as the subject information. Therefore, in the present embodiment, as shown in FIG. 8, the test order acquisition unit 62 acquires an order list 72 of the test order associated with the name, gender, age, and ID of the subject 10. Each row in the order list 72 is a test order.

The label recognition unit 63 recognizes the subject 10 from whom the sample contained in each sample container 13 is collected by using the label image 55. Specifically, the label recognition unit 63 is a processor or the like that executes so-called optical character recognition/reader (OCR) software. “Recognizing the subject 10” means acquiring at least one piece of subject information to the extent that the subject 10 can be specified. For example, the label recognition unit 63 recognizes the subject name, gender, and/or age described on the label 35. Further, since the subject name, gender, and/or age described on the label 35 are described in characters or symbols, the label recognition unit 63 recognizes these characters or symbols by reading them and converting them into characters or symbols that can be used in the management system 80 (character codes or the like used in the management system 80). In the present embodiment, the label recognition unit 63 recognizes the name, gender, and age of the subject. This is to recognize each subject 10 more accurately. The subject name is the “surname” and/or “name” of the subject 10 (including the middle name and the like).

Further, the label recognition unit 63 can recognize the color of the label 35 by using the label image 55. Recognizing the color of the label 35 means specifying whether the color of each character or the like indicating the items described on the label 35 is the first color or the second color. This is to distinguish two days' worth of samples in the fecal occult blood test. In addition, the label recognition unit 63 can recognize the subject 10 by using the label image 55 of the label 35 handwritten by the subject 10. This is because the items described on the label 35 are often handwritten by the subject 10.

The automatic association unit 65 automatically associates the test result related to the sample of each sample container 13 with the test order acquired by the test order acquisition unit 62 by using the recognition result of the label recognition unit 63. The “association” between the test result and the test order means specifying the test result related to the sample of a specific sample container 13 as the test result related to a specific test order, and is substantially synonymous with specifying a specific sample container 13 as the sample container 13 related to a specific test order and/or identifying that a specific subject 10 who provided the sample of a specific sample container 13 is the subject 10 related to a specific test order. This is because in a case where one of these is associated, the other is automatically determined. Further, since the sample container 13 has a one-to-one correspondence with the label image 55, the association between the sample container 13 and the test order can be rephrased as the association between the label image 55 and the test order.

In the present embodiment, the automatic association unit 65 collates a part or all of the recognition result of the label recognition unit 63 with the corresponding item of the test order. Then, as a general rule, the test order in which contents of the subject information included in the recognition result and the test order in common match each other is associated with the label image 55 that has performed the recognition process and the record of the measurement result (“measured value”) (record of the specific S/N in the test data 71 of FIG. 7). In addition, in a case where there is no test order in which the contents of the subject information included in the recognition result and the test order in common match each other, a test order having a high match rate among the partially matching test orders is associated with the set of the label image 55 that has performed the recognition process and the measurement result. That is, as a result of the collation between the recognition result of the label image 55 and the test order, the automatic association unit 65 presumes that the test order having a good match rate (for example, the highest match rate) of the subject information is the test order corresponding to the set of the label image 55 that has performed the recognition process and the measurement result to perform automatic association. As a result, even in a case where the label image 55 is poorly recognized, the automatic association unit 65 can perform automatic association that is tentatively considered to be the most accurate.

Further, in a case where the label recognition unit 63 recognizes a plurality of pieces of subject information, the automatic association unit 65 may set a priority among the pieces of subject information. For example, in a case where the label recognition unit 63 recognizes the name, gender, and age of the subject 10 from the label image 55, the automatic association unit 65 prioritizes the name of the subject 10 among these information and associates the name with the test order. This is because the probability that the name of the subject 10 matches the other subject 10 is lower than the probability that the gender and/or age match the other subject 10, so that by giving priority to the degree of matching of names, it is easy to make an accurate association even in a case where the subject information does not completely match.

The display unit 66 displays the test result and the test order in association with each other according to association by the automatic association unit 65. This is because the automatic association unit 65 can accurately perform the above association, but an operator of the management device 22 should be requested to make a final check and approval. The operation unit 67 is, for example, a pointing device or the like, and is used for the above approval operation.

Of the test device 21 and the management device 22, the label imaging unit 52 of the test device 21 and a combination of at least some (the test order acquisition unit 62, the label recognition unit 63, the automatic association unit 65, and the display unit 66) of the management device 22 constitutes the management system 80 (see FIG. 5). The management system 80 is a system that acquires the label image 55 and automatically associates a test result with a test order by using the label image 55.

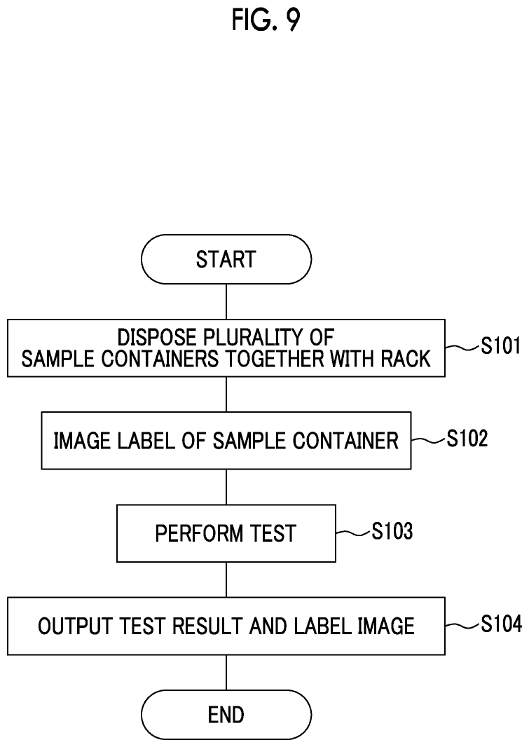

Hereinafter, the operation related to the test and the association with the test order will be described. First, as shown in FIG. 9, in a case where the testing institution 12 receives a request for the test for a plurality of persons from the hospital 11 and receives the sample containers 13 containing a sample, a staff of the testing institution 12 sets the plurality of sample containers 13 in the rack 46 by aligning the surfaces to which the label 35 is attached. (Step S101). Then, the plurality of sample containers 13 are placed on the sample container placing portion 41 of the test device 21 together with the rack 46. As a result, the test device 21 enters a state where the test can be started by inputting a test start instruction.

After that, in a case where a test start instruction is input using the touch panel 43, the label imaging unit 52 images the label 35 attached to the sample container 13 and obtains the label image 55 (Step S102). On the other hand, the fecal occult blood test mechanism 53 acquires a sample from the sample container 13 in which the label 35 is imaged, and performs a fecal occult blood test (Step S103). Then, the test unit 42 sequentially outputs the label image 55 acquired by the label imaging unit 52 and the test result output by the fecal occult blood test mechanism 53 to the management device 22 in a one-to-one correspondence (Step S104). By automatically repeating these steps, the test device 21 automatically tests the samples in the plurality of set sample containers 13.

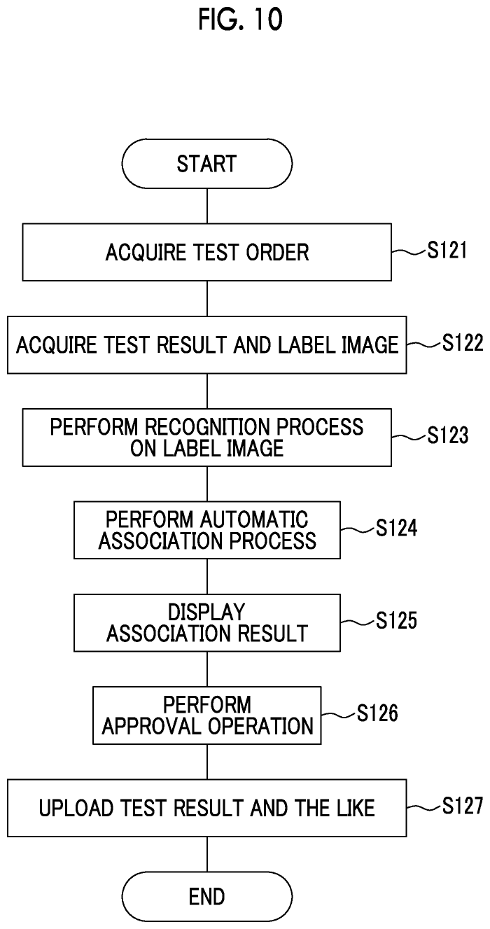

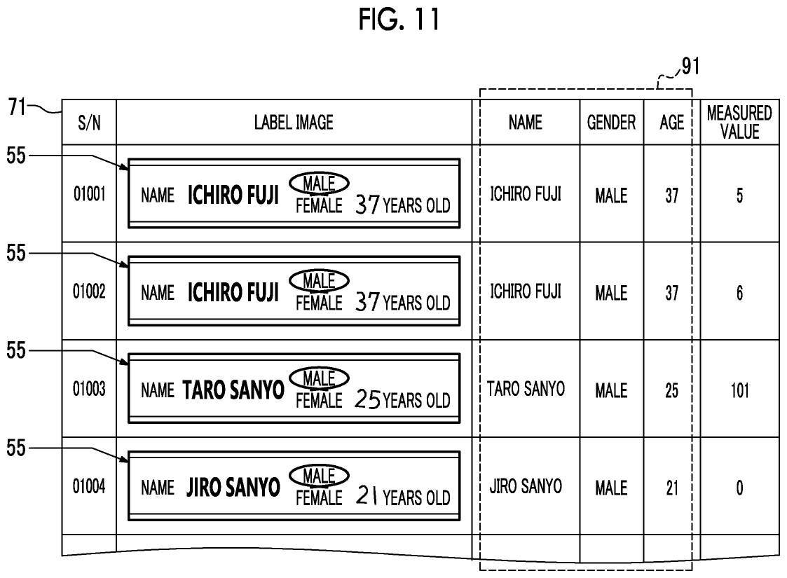

In the management device 22, as shown in FIG. 10, the test order acquisition unit 62 acquires the test order from the hospital information system 16 or the like in advance (Step S121). As a result, the management device 22 obtains the order list 72. On the other hand, in a case where the test result acquisition unit 61 acquires the test result and the label image 55 from the test unit 42 of the test device 21 (Step S122), the label recognition unit 63 performs a recognition process on the label image 55 (Step S123), and recognizes the subject 10 from whom the sample contained in the sample container 13 is collected. As a result, in the present embodiment, the label recognition unit 63 obtains information on the name, gender, and age of the subject 10. Therefore, as shown in FIG. 11, a field (hereinafter, referred to as a recognition result field) 91 of the name, gender, and age of the subject 10, which is the recognition result of the label recognition unit 63, is added to the test data 71 held by the management device 22.

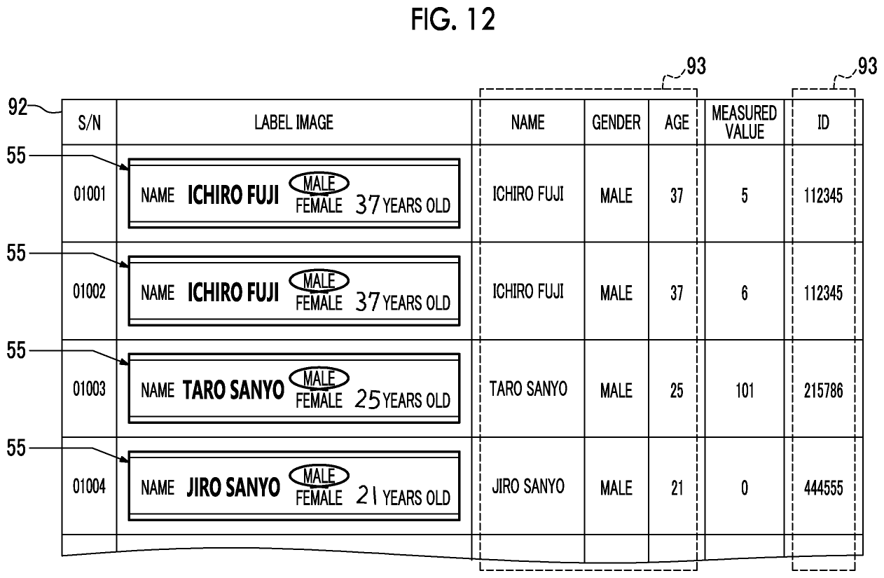

After that, the automatic association unit 65 automatically associates each test result with the test order by collating the data in the recognition result field 91 of the test data 71 with the data in each row of the order list 72 (Step S124). Then, the display unit 66 displays the test data 71 and the test order in association with each other according to the association by the automatic association unit 65 (Step S125). More specifically, as shown in FIG. 12, the display unit 66 displays associated data 92 in which the test data 71 and the test order are integrated according to the result of the association. The associated data 92 displays, for example, items such as a S/N, a label image, the name, gender, and age of the subject 10, a measured value which is the test result, and an ID of the subject 10. For example, the items such as the S/N, the label image, and the measured value among these are the data of the test data 71, and data in a field (hereinafter, referred to as order data field) 93 of the items such as the name, gender, age, and ID of the subject 10 is the data in the order list 72.

In a case where the test is completed and the display unit 66 displays the associated data 92, the staff of the testing institution 12 checks the match of the contents of the label image 55 and the contents of the order data field 93 (particularly, the name, gender, and age fields), for example, and then performs an approval operation using the operation unit 67 (Step S126). For example, on the screen of the display unit 66, an “approval button” (not shown) is pressed. As a result, the management device 22 reports the measured value, which is the test result, as the test result of the corresponding test order. For example, the management device 22 can register (upload) the test result in a system in the hospital 11 such as the hospital information system 16.

As described above, according to the test device 21 and the management device 22, particularly the part constituting the management system 80 (hereinafter, referred to as the management system 80 and the like), the testing institution 12 can automatically test the samples contained in the plurality of sample containers 13 and automatically associate the results with the test order. For example, in a typical testing institution 12 in the related art, in order to identify a plurality of sample containers 13, it is necessary to give a barcode or the like to each sample container 13, create data for associating an ID given by the barcode or the like with the subject 10 who submitted the sample container 13, and obtain a test result while collating the data with a plurality of test orders. On the other hand, according to the management system 80 and the like, in a case where the plurality of sample containers 13 are set in the rack 46, the above association can be automatically, quickly, and accurately performed simply by aligning the surfaces to which the label 35 is attached. Therefore, according to the management system 80 and the like, the efficiency and accuracy of the work in the testing institution 12 can be improved.

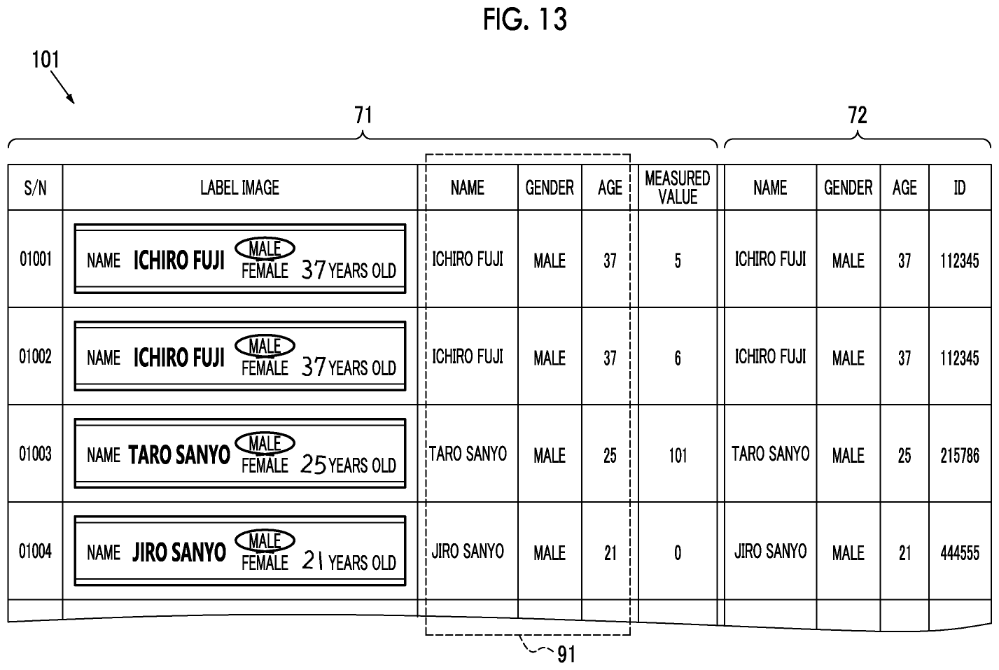

In the first embodiment, the display unit 66 displays the associated data 92 in which the test data 71 and the test order are integrated, but instead of integrating in this way, as shown in FIG. 13, the display unit 66 can display associated data 101 in which the test data 71 and the test order are associated with each other without breaking the arrangement and the like of the test data 71 and the data in the order list 72. In the associated data 101, not only the label image 55 but also the recognition result shown in the recognition result field 91 are directly compared with the test order, and it is possible to check whether or not the association by the automatic association unit 65 is accurate.

Further, in the first embodiment, the label recognition unit 63 can recognize the color of the label 35 by using the label image 55, and the recognition result related to the color of the label 35 can be used for associating the sample container 13 with the test order. For example, in a case where the label recognition unit 63 can recognize the color of the label 35, and the label recognition unit 63 or the automatic association unit 65 can check by comparing the description contents of the labels 35 of different colors that they are the corresponding labels 35 of different colors, the automatic association unit 65 may preferentially associate the same test order for two sample containers 13 having the label images 55 of different colors. This is because even in a case where one of the labels 35 of different colors has poor recognition due to stains, entry errors, forgetting to enter, or the like, accurate association can be easily performed. It is particularly useful in fecal occult blood tests using the labels 35 of different colors.

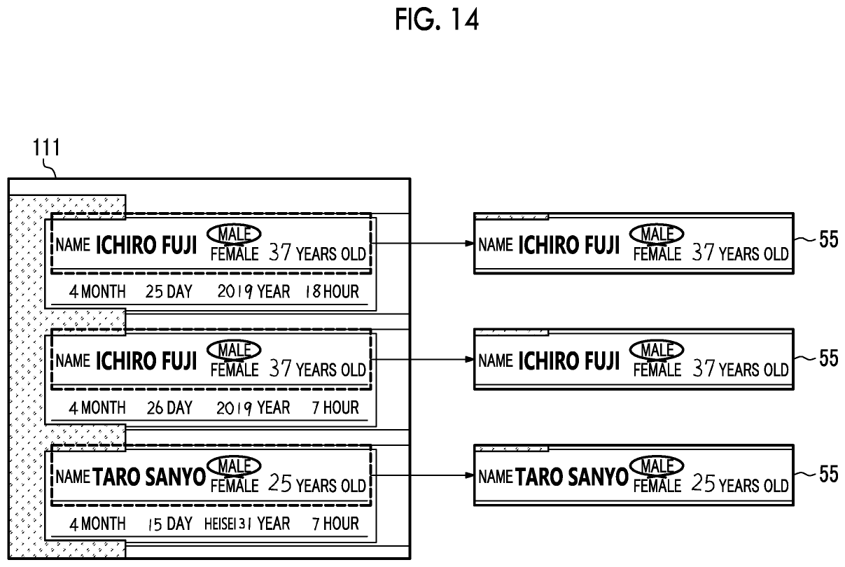

Further, in the first embodiment, the label imaging unit 52 images the label 35 of each sample container 13, but as shown in FIG. 14, the label imaging unit 52 can obtain the label image 55 by imaging the labels 35 of the plurality of sample containers 13 together and extracting a portion of the label 35 of each sample container 13 as the label image 55 from an obtained image 111. In this way, in a case where the label image 55 is individually extracted from the image 111 obtained by imaging the labels 35 of the plurality of sample containers 13 together, the number of times the label 35 is imaged can be reduced. In a case where the number of sample containers 13 is particularly great and the imaging of the label 35 causes a long time required for the test, the test time including the imaging of the label 35 can be shortened.

[Second Embodiment]

In the first embodiment, the label recognition unit 63 recognizes the form of the subject information described on the label 35 by using characters or symbols as it is, and the automatic association unit 65 associates the sample container 13 with the test order by using the form of the characters or symbols recognized by the label recognition unit 63 as it is. However, even though the subject information of the same subject 10 is used, the form of characters or symbols may differ in a part or all of the label 35 of the sample container 13 and the test order. For example, the name of the subject is registered in “Kanji” in the test order, and the subject describes the name in “Hiragana” or “Katakana” on the label 35.

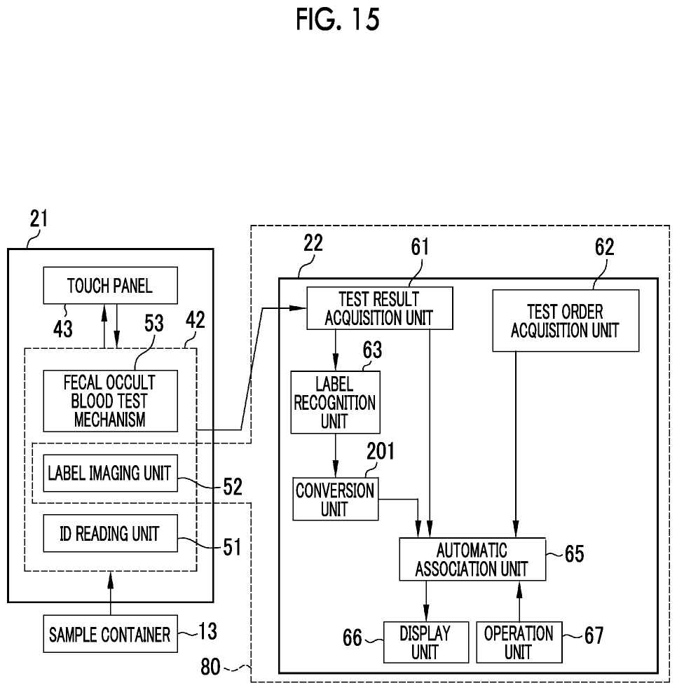

In order to handle the above situation, as shown in FIG. 15, the management system 80 can comprise, for example, a conversion unit 201 in the management device 22. The conversion unit 201 converts the character or symbol recognized by the label recognition unit 63 into another character or symbol. For example, in a case where the management system 80 is used in Japan, the conversion unit 201 can convert, for example, “Kanji”, “Hiragana”, “Katakana”, “Romaji” (alphabet), and the like to each other. Further, the conversion performed by the conversion unit 201 includes conversion of “full-width” and “half-width”. In a case where the management system 80 is used in a language area other than Japanese, the conversion unit 201 is configured to convert characters or symbols between a plurality of types of characters used in the area where the management system 80 is used. For example, in the Chinese-speaking area, the conversion unit 201 can be configured to perform mutual conversion between “Kanji” and “Pinyin”.

In a case where the management system 80 comprises the conversion unit 201, the automatic association unit 65 associates each test result with the test order by using a character or a symbol obtained by converting the recognition result of the label recognition unit 63 by the conversion unit 201. This is because the accuracy of the association is improved.

It is preferable that the conversion unit 201 converts the character or symbol recognized by the label recognition unit 63 into the character or symbol used in the test order for recording the subject information. This is to improve the accuracy of the association process in the automatic association unit 65 in a case where different types of characters or the like are used for the label 35 and the test order. In particular, it is preferable that the conversion unit 201 converts the character recognized by the label recognition unit 63 into the character type used in the test order for the subject name. This is because the subject name is particularly useful for identifying each subject in the subject information. In this case, the conversion unit 201 acquires in advance information related to the type of characters or symbols used for the subject information in the test order based on the test order.

[Third Embodiment]

The label recognition unit 63 may not be able to correctly recognize the subject information from the label image 55. For example, in a case where a part or all of the label 35 is stained, or in a case where the subject 10 himself/herself writes the subject information on the label 35, the characters or the like are messy. In such a case, it is preferable that the management system 80 of the first embodiment and/or the second embodiment accepts corrections for the recognition result of the label recognition unit 63 and/or the result of the association process of the automatic association unit 65, particularly for the subject information of the subject 10 recognized by using the label image 55 with poor recognition. Finally, this is because it is necessary to accurately associate the test result with the test order.

In a case where corrections for the recognition result of the label recognition unit 63 and/or the result of the association process of the automatic association unit 65 are accepted, for example, the label recognition unit 63 is configured to output the recognition result and the accuracy indicating the certainty of the recognition result. Then, it is preferable that the display unit 66 highlights and displays a display portion in which the test result related to the label image 55 with poor recognition including a portion whose accuracy of the recognition process is equal to or less than a threshold value and the test order are associated with each other. This is to prompt a check in the associated data 92.

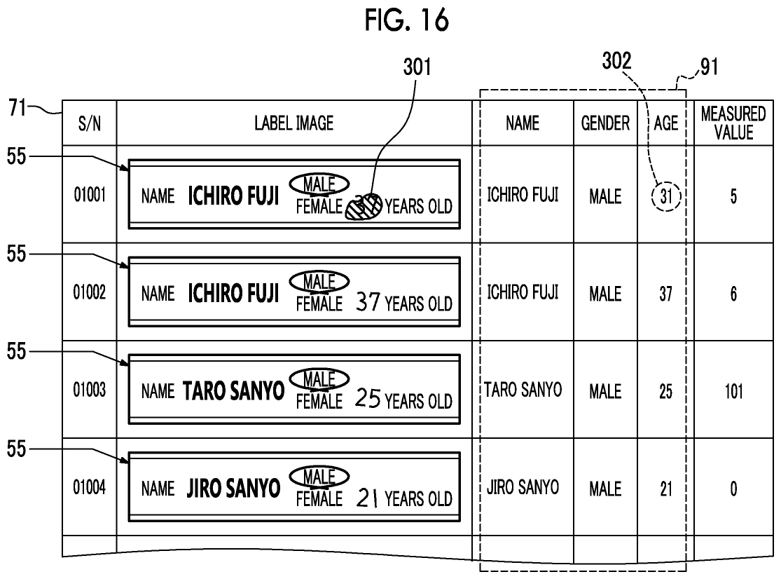

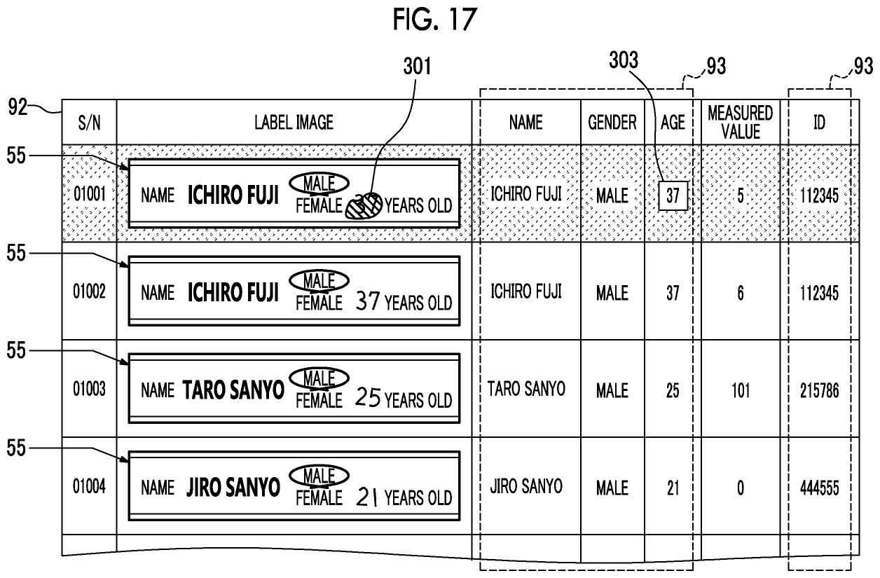

For example, as shown in FIG. 16, it is assumed that there is a stain 301 in the age entry field 38 of the label image 55 of a S/N “01001”, and as shown in an age portion 302, the label recognition unit 63 erroneously recognizes the age of the subject 10 as “31”, for example. In this way, in a case where there is a stain 301 or the like in a part or all of the label image 55, the accuracy output by the label recognition unit 63 is equal to or less than a predetermined threshold value. Therefore, as shown in FIG. 17, the display unit 66 highlights and displays a row of the associated data 92 to which the association is performed using the recognition result whose accuracy is equal to or less than the threshold value, and/or a portion 303 related to the recognition result whose accuracy is equal to or less than the threshold value. In this way, in a case where the row (record) to which the association is performed using the recognition result whose accuracy is equal to or less than the threshold value and/or the portion 303 is highlighted, since a checking person can easily specify the row or the portion 303 that requires a special check from a plurality of pieces of data of the associated data 92, it can be corrected or approved without being overlooked. Similarly, in a case where the associated data 101 of the form shown in FIG. 13 is displayed, it is possible to highlight and display the data.

In a case where the recognition result by the label recognition unit 63 includes a recognition result whose accuracy is equal to or less than the threshold value, the display unit 66 can display the test data 71 and request the checking person to correct the recognition result itself before the automatic association unit 65 performs the automatic association process. In this case, similarly to the associated data 92 (FIG. 17), it is preferable to highlight and display the portion requiring a check and/or the row (record) including the portion requiring a check. This is for the checking person to correct or approve the relevant portion without overlooking it by clearly indicating the portion for which a check is requested.

Further, in the first embodiment, the second embodiment, and the third embodiment, the display unit 66 displays the label image 55 in the display of the associated data 92 and the like, but it is preferable that the display unit 66 displays the label image 55 for at least the subject 10 recognized by using the label image 55 with poor recognition. This is for the checking person to collate the data with the label image 55 and correct it correctly.

It is preferable that in a case where the checking person corrects the subject information of the subject 10, the automatic association unit 65 automatically associates the test order with the test result by using the corrected subject information. This is because an accurate association result can be easily proposed by automatically performing the association process using the corrected subject information. In addition, it is efficient because the manual association work by the checking person can be omitted.

Further, at least in a case where there is the label image 55 with poor recognition, the automatic association unit 65 may present a candidate for the test order that can be associated with the test result in addition to the test order that is automatically associated with the test result. This is because in a case where a candidate for the test order is presented, the checking person can easily correct the association result simply by selecting an appropriate test order from the presented candidate.

In the first embodiment, the second embodiment, the third embodiment, and each modification example, the management system 80 is composed of the label imaging unit 52 of the test device 21, the test order acquisition unit 62, the label recognition unit 63, the automatic association unit 65, and the like of the management device 22, but each unit constituting the management system 80 can be incorporated into any device. For example, in a case where all the units constituting the management system 80 are provided in the test device 21, the test device 21 itself functions as the management system 80. In this case, the touch panel 43 can be used as the display unit 66. Further, in the first embodiment, the second embodiment, the third embodiment, and each modification example, the label imaging unit 52 is incorporated in the test device 21, but the label imaging unit 52 may be provided in the management device 22. In this case, since the management device 22 includes all the units constituting the management system 80, the management device 22 itself functions as the management system 80 in the above-described embodiment and the like. Further, the label imaging unit 52 can be provided as a part other than the test device 21 and the management device 22. For example, the label imaging unit 52 can use a digital camera or the like that is driven independently of the test device 21 and the management device 22.

In each of the above embodiments and modification example, it is preferable that the test device 21, the management device 22, and/or the management system 80 (hereinafter, referred to as the test device 21 or the like) have the following function or operation mode. The following function or operation mode is particularly useful for a test device 21 or the like (a so-called calibration-free test device 21 or the like) in which calibration is rarely performed.

<Identification of Administrator and Operator by ID>

The test device 21 or the like can identify the operator by an ID or the like and limit or release the function or operation mode that can be used by the operator's ID or the like. For example, the test device 21 or the like identifies two types of IDs: an administrator (ADM) who has the management authority to change the settings of the test device 21 or the like; and an operator (OPE) who has the use authority but does not have the management authority for the test device 21 or the like. In a case where there are a plurality of administrators in the test device 21 or the like, the test device 21 or the like gives an administrator ID to each of the plurality of administrators and identifies each administrator. Similarly, in a case where there are a plurality of operators in the test device 21 or the like, the test device 21 or the like issues an operator ID to each of the plurality of operators and identifies each operator. In a case where the management authority can be used simply by entering a password or the like, it is unknown who changed the device setting or the like and who instructed the execution of the test or the like. On the other hand, as described above, the administrator and the operator can be identified by an ID or the like, and the person who has set the test device 21 or the like or executed the test can be specified. It is possible to easily contact or inquire of the administrator who changed the device setting or the operator who executed the test, as necessary.

Further, in a case where neither the administrator's ID nor the operator's ID is input, the test device 21 or the like prohibits the execution of test or other operations. The administrator inputs the ID into the test device 21 or the like, so that the test device 21 or the like provides an operation screen (graphical user interface (GUI)) for an administrator or enables other operation units such as buttons to be used. For example, a GUI button or the like related to device setting of the test device 21 or the like and a GUI button or the like for inputting a test execution instruction to the test device 21 or the like are displayed. As a result, the administrator can execute the test or the like by using the test device 21 or the like, and can further perform the device setting the test device 21 or the like, the authority setting for each ID (setting such as giving the management authority to a specific operator), and the operation mode setting such as giving a limit on the number of times of use to a specific operation mode, depending on the administrator authority. The operator inputs the ID into the test device 21 or the like, so that the test device 21 or the like provides an operation screen for an operator or enables other operation units such as buttons to be used within the range necessary for the test and the like. For example, a GUI button or the like related to the device setting of the test device 21 or the like is not displayed and a GUI button or the like for inputting a test execution instruction to the test device 21 or the like is displayed. As a result, the operator can execute the test or the like using the test device 21 or the like.

<Non-Use or Non-Display Function of Some Test Results>

Consumables (reagents and the like) used for a test in the test device 21 usually have an expiration date. Therefore, the test device 21 can prohibit the test using the consumables whose expiration date has passed (hereinafter, referred to as overdue consumables). Further, in a case where the test device 21 does not prohibit the test using the overdue consumables, the test device 21 or the like can set, as non-use or non-display, the result of the test using the overdue consumables. As a result, it is possible to prevent the use of inappropriate test results. Although the test using overdue consumables has been described here, some test results can be set as non-use or non-display based on other factors. In addition, “setting as non-use” of the test result means avoiding (or prohibiting) the use of the test result, such as not inputting it to the test result acquisition unit 61 or not using it for automatic association. “Setting as non-display” the test result includes not displaying the test result on the touch panel 43 and/or the display unit 66, and concealing the test result by displaying “expired” or “expire (EXP)” on a portion where the test result should be displayed.

<Emergency Mode>

As described above, it is preferable that in a case where the result of the test using the overdue consumables is set as non-use or non-display, the test device 21 or the like has an emergency (EMG) mode as one of the operation modes. The emergency mode is an operation mode for forcibly using or displaying a test result that is set as non-use or non-display, such as the result of the test using overdue consumables. This is to prepare for an emergency where it is advantageous to use or display the test result, even considering the possibility that the test result may be inaccurate. It is preferable that in a case where the emergency mode is provided, the administrator sets a limit on the number of times the emergency mode can be used. This is to prevent abuse of the emergency mode. In a case where the emergency mode reaches the upper limit of the number of times of use, the test device 21 or the like does not use or display the result or the like of the test using the overdue consumables.

<Quality Control (QC) Function>

It is preferable that the test device 21 has a quality control function as one of the operation modes. The quality control function is a function of controlling test quality by determining and displaying the accuracy of the test in, for example, periodic inspection of the test device 21 or the like. For example, in a case of using the quality control function, a simulated test is performed using consumables and the like for quality control, and the test device 21 or the like determines and displays the quality of the test result according to a standard set for the quality control function (for example, a standard of OK or NG). In addition, the operator or the administrator of the test device 21 or the like can visually highlight and display whether the test result belongs to OK or NG by setting the judgment standard of OK or NG for the test results of the actual sample separately from the standard for quality control.

<Unusable Setting for Consumables, etc.>

It is preferable that the test device 21 or the like can set consumables and the like used for the test as consumables that cannot be used. For example, by registering the production lot number or serial number of overdue consumables and the like, and setting it to be unusable, it is possible to prevent accidentally executing a test using overdue consumables and the like (for example, without noticing that overdue consumables are set). In addition, in a case where a manufacturing defect is found in the consumables used for a test, and the registration and the unusable setting are made as described above, it is possible for the test device 21 or the like to automatically exclude the execution of the test using the corresponding consumables without artificially searching for the corresponding consumables. Here, consumables (reagents and the like) used for the test are taken as an example, but it is preferable that the sample container 13 can also be registered and set as unusable as necessary.

In each of the above embodiments and modification example, a test device 21 for performing a fecal occult blood test using stool is used, but as the test device 21, a test device for performing a test other than a fecal occult blood test using stool, a blood test device for performing a test using blood, or a device for performing a test using other tissues (mucosal cells or the like), mucus, or the like can be optionally used. Therefore, the management system 80 is effective for various test devices or combinations of test devices.

In each of the above embodiments and modification example, the hardware structure of the processing unit that executes various processes such as the label recognition unit 63 and the automatic association unit 65 is various processors as shown below. The various processors include a central processing unit (CPU) that is a general-purpose processor that functions as various processing units by executing software (program), a programmable logic device (PLD) that is a processor whose circuit configuration can be changed after manufacture, such as a graphical processing unit (GPU) or a field programmable gate array (FPGA), a dedicated electrical circuit that is a processor having a circuit configuration designed exclusively for executing various types of processing, and the like.

One processing unit may be configured by one of various processors, or may be configured by a combination of the same or different kinds of two or more processors (for example, a combination of a plurality of FPGAs, a combination of a CPU and an FPGA, a combination of a CPU and a GPU, or the like). In addition, a plurality of processing units may be configured by one processor. As an example of configuring a plurality of processing units by one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more of the above-described various processors as hardware structures.

More specifically, the hardware structure of these various processors is an electrical circuit (circuitry) in the form of a combination of circuit elements, such as semiconductor elements.

### Explanation of References

10: subject

11: hospital

12: testing institution

13: sample container

16: hospital information system

21: test device

22: management device

31: container body

32: cap

35: label

36: name entry field

37: gender selection field

38: age entry field

39: stool collection date and time entry field

41: sample container placing portion

42: test unit

43: touch panel

46: rack

51: ID reading unit

52: label imaging unit

53: fecal occult blood test mechanism

55: label image

61: test result acquisition unit

62: test order acquisition unit

63: label recognition unit

65: automatic association unit

66: display unit

67: operation unit

71: test data

72: order list

80: management system

91: recognition result field

92: associated data

93: order data field

101: associated data

111: image

201: conversion unit

301: stain

302: age portion

303: portion

S101 to S127: operation steps

What is claimed is:

1. A management system comprising:
an imaging device configured to image labels attached to a plurality of sample containers each containing a sample, the label having subject information of a subject from whom the sample is collected described thereon;
a processor configured to function as:
a label recognition unit configured to recognize the subject from whom the sample contained in each of the sample containers is collected by using label images obtained by imaging the labels;
a test order acquisition unit configured to acquire a test order including the subject information of the subject; and
an automatic association unit configured to automatically associate a test result related to the sample of each sample container with the test order acquired by the test order acquisition unit by using a recognition result of the label recognition unit; and
a display configured to display the test result and the test order in association with each other according to association by the automatic association unit.

2. The management system according to claim 1, wherein
the label recognition unit recognizes a color of the label by using the label image, and
the automatic association unit associates the same test order for two sample containers having the label images of different colors.

3. The management system according to claim 1, wherein the imaging device images the labels of the plurality of sample containers together, and extracts a portion of the label of each sample container as the label image from an obtained image.

4. The management system according to claim 1, wherein
description of the label includes a subject name, gender, and/or age of the subject, and
the label recognition unit recognizes the subject name, gender, and/or age described on the label.

5. The management system according to claim 1, wherein the processor is further configured as function as:
a conversion unit configured to convert a character or a symbol recognized by the label recognition unit into another character or symbol,
wherein the automatic association unit associates each test result with the test order by using a character or a symbol obtained by converting the recognition result of the label recognition unit by the conversion unit.

6. The management system according to claim 5, wherein the conversion unit converts the character recognized by the label recognition unit into a character type used in the test order for a subject name.

7. The management system according to claim 1, wherein the label recognition unit recognizes the subject by using the label image of the label handwritten by the subject.

8. The management system according to claim 1, wherein the display highlights a display portion in which the test order and the test result related to the label image with poor recognition whose accuracy of a recognition process is equal to or less than a threshold value are associated with each other.

9. The management system according to claim 8, wherein the display displays the label image for at least the subject recognized by using the label image with poor recognition.

10. The management system according to claim 8, wherein a correction for the subject information of the subject recognized by using the label image with poor recognition is accepted.

11. The management system according to claim 10, wherein in a case where the subject information of the subject is corrected, the automatic association unit automatically associates the test order with the test result by using the corrected subject information.

12. The management system according to claim 8, wherein at least in a case where there is the label image with poor recognition, the automatic association unit presents a candidate for the test order that is able to be associated with the test result in addition to the test order that is automatically associated with the test result.

13. The management system according to claim 1, wherein the display integrally displays the test result and the test order associated each other by the automatic association unit.
